(19) United States
(12) Patent Application Publication
(10) Pub. No.: US 2017/0102306 A1
(43) Pub. Date: Apr. 13, 2017

(54) ABSORBENT ARTICLE MADE OF TOW FIBERS AND SAP ADDING OPEN FORMATION HIGH LOFT LAYER WITH FIBERS ORIENTED IN Z DIRECTION TO IMPROVE SAP STABILITY

(71) Applicant: INDEVCO Ltd., Beirut (LB)

(72) Inventors: Georges DAGHER, Beirut (LB); Georges MOUFARREJ, Beirut (LB)

(73) Assignee: INDEVCO LTD., Beirut (LB)

(21) Appl. No.: 14/879,403

(22) Filed: Oct. 9, 2015

(51) Int. Cl. G01N 5/02; A61F 13/534

(52) U.S. Cl. CPC G01N 5/025; A61F 13/534; A61F 2013/530226

(57) ABSTRACT

An absorbent article, including a liquid permeable top sheet, a pulpless absorbent core, the core including synthetic fibers and a super absorbent material, the super absorbent material being settled and stabilized by an open formation bottom high loft layer with fibers oriented in a Z direction, and a liquid impermeable backsheet, wherein the absorbent core is positioned between the top sheet and the backsheet.

### BACKGROUND

Mass production of disposable diapers began in the 1960s. The early diaper cores consisted of 100% fluff pulp. The performance of these early cores was not sufficiently efficient, as the liquid absorbed by the fluff pulp was easily expressed from the structure upon application of pressure to a saturated core. The introduction of super absorbent polymers (SAP) in the early 1980s allowed for higher performance diaper cores. The use of SAP allowed for the reduction or complete removal of the bulky fluff pulp from the absorbent core. This allowed the diapers to become thinner while maintaining an improved performance. The reduction in physical s was a desired feature for consumers and also led to savings in handling and transportation of such products.

Absorbent products such as baby diapers, feminine hygiene pads and adult incontinence products are typically constructed of several different types of materials. These products typically include a permeable non-woven top sheet, an impermeable back sheet and an absorbent core sandwiched therebetween. The absorbent core typically consists of either wood fluff or synthetic fiber, e.g a TOW, and a liquid-absorbing polymer, e.g. a SAP.

The super absorbent polymers are typically based on acrylic acid and sodium acrylate, and are able to swell so as to absorb and retain a quantity of liquid several times the weight of the SAP, consequently forming a gel. The gel formation from the absorbed liquid allows the SAP to act as a fluid locking system, even under pressure.

The TOW fibers are a continuous “rope” of fibers consisting of many filaments loosely joined side-to-side. Filament is a continuous strand consisting of anything from 1 filament to many. Synthetic fiber is most often measured in a weight per linear measurement basis, along with cut length. Denier and Dtex are the most common weight to length measures. Cut-length only applies to staple fiber. Those fibers are oriented in machine direction and are capable of spontaneously transporting liquid on their surfaces. Presently available absorbent articles such as diapers, sanitary napkins, incontinence briefs, and the like are generally very good at absorbing aqueous fluids such as urine and blood. Generally with TOW in a diaper or similar article, which starts and stops at the ends of the diaper, or a staple fiber of some specified cut length, the ability to move fluid ceases once the fluid reaches the ends of the fibers unless “sinks” for the fluid are provided.

A weakness of TOW and SAP wrapped with tissue or nonwoven technology is bad SAP stability, which may lead to some areas with no SAP and other areas with high concentrated SAP, both of which may lead to gel blocking and leakage.

### SUMMARY

According to at least one exemplary embodiment, an absorbent article is disclosed. The absorbent article can include a liquid permeable top sheet, a pulpless absorbent core, the core including synthetic fibers (TOW) and an open formation high loft layer with fibers oriented in Z direction wherein a super absorbent material is disposed therebetween. The absorbent article may further include a liquid impermeable backsheet. The absorbent core may be situated between the top sheet and the backsheet.

### BRIEF DESCRIPTION OF THE FIGURES

Advantages of embodiments of the present invention will be apparent from the following detailed description of the exemplary embodiments. The following detailed description should be considered in conjunction with the accompanying figures in which:

FIG. 1A is a top view of an exemplary embodiment of an absorbent article having an absorbent core.

FIG. 1B is a top view of an exemplary embodiment of an absorbent article having an absorbent core.

FIG. 1C is a top view of an exemplary embodiment of an absorbent article having an absorbent core.

FIG. 2A is a top sectional view of an exemplary embodiment of a high loft layer.

FIG. 2B is a cross sectional view of an exemplary embodiment of a high loft layer.

FIG. 2C is a cross sectional view of exemplary embodiment of an absorbent core.

FIG. 3 is a cross-sectional view of an exemplary embodiment of an absorbent core, illustrating positioning and stabilization of the super absorbent polymer within the core.

FIG. 4A shows an exemplary absorbent core wrap configuration.

FIG. 4B shows an exemplary absorbent core wrap configuration.

FIG. 4C shows an exemplary absorbent core wrap configuration.

FIG. 5 illustrates an exemplary embossed pattern for an absorbent synthetic core.

FIG. 6 is a diagram of an exemplary absorbent core forming unit for an absorbent article.

FIG. 7 illustrates an exemplary zoning testing for measuring the super absorbent polymer SAP distribution in the product.

### DETAILED DESCRIPTION

Aspects of the invention are disclosed in the following description and related drawings directed to specific embodiments of the invention. Alternate embodiments may be devised without departing from the spirit or the scope of the invention. Additionally, well-known elements of exemplary embodiments of the invention will not be described in detail or will be omitted so as not to obscure the relevant details of the invention. Further, to facilitate an understanding of the description discussion of several terms used herein follows.

As used herein, the word “exemplary” means “serving as an example, instance or illustration.” The embodiments described herein are not limiting, but rather are exemplary only. It should be understood that the described embodiments are not necessarily to be construed as preferred or advantageous over other embodiments. Moreover, the terms “embodiments of the invention”, “embodiments” or “invention” do not require that all embodiments of the invention include the discussed feature, advantage or mode of operation.

Embodiments disclosed herein relate to absorbent products, such as disposable diapers, incontinence pads, sanitary napkins, and the like, which have an absorbent core that includes synthetic fibers and super absorbent material, such as super absorbent polymers (SAP), settled in loft layer holes. Such an absorbent material may simultaneously optimize SAP stabilization as well as permeability, thereby providing improved absorption, rewetting and comfort. In an exemplary embodiment, the loft layer may be a high loft layer made of a low density, thick fabric. The high loft layer may have a high ratio of thickness to weight per unit area. In at least some exemplary embodiments, high loft battings may have no more than 10% solids by volume and may be thicker than 3 mm (0.13 inches).

The inclusion of an open formation high carrying loft layer with fibers oriented in a Z direction at the bottom side of the absorbent core may allow the SAP to settle inside the voids and between the fibers oriented in a Z direction of the open formation high loft layer and the TOW fibers. This may improve the SAP fixation and stability to a uniform core. The fibers may be bonded together with spray hot melt adhesive. In such an exemplary embodiment, the SAP may have the advantageous ability to migrate in a Z direction toward the bottom of the diaper.

An exemplary embodiment may lead to better leakage performance and SAP migration thru the core, avoiding SAP blocking. The high loft layer may further provide improved softness or comfort.

According to at least one exemplary embodiment, about 60% to about 100% of the SAP can be attached between the synthetic fibers and the open formation high loll layer with fibers oriented in a Z direction.

According to at least one exemplary embodiment, the absorbent core can be made from about 50% to about 95% of super absorbent polymer SAP settled inside the voids and between the fibers oriented in a Z direction of the open formation high loll layer. The developed SAP material stability can improve the liquid spreading and can prevent excessive rewetting and leakage.

According to at least one exemplary embodiment, an absorbent article can include an absorbent core having a super absorbent polymer SAP settled inside the voids and between the fibers oriented in a Z direction of the open formation high loft layer and the synthetic fiber TOW. The absorbent core may be pulpless. The absorbent article structure can result in a thin product. Such absorbent articles can include disposable diapers, incontinence pads, sanitary napkins, and the like, wherein the absorbent core includes synthetic fibers and super absorbent polymer SAP settled inside the voids and between the fibers oriented in Z direction of the open formation high loft layer. The SAP can be between about 50% to about 95% of the weight of the absorbent core.

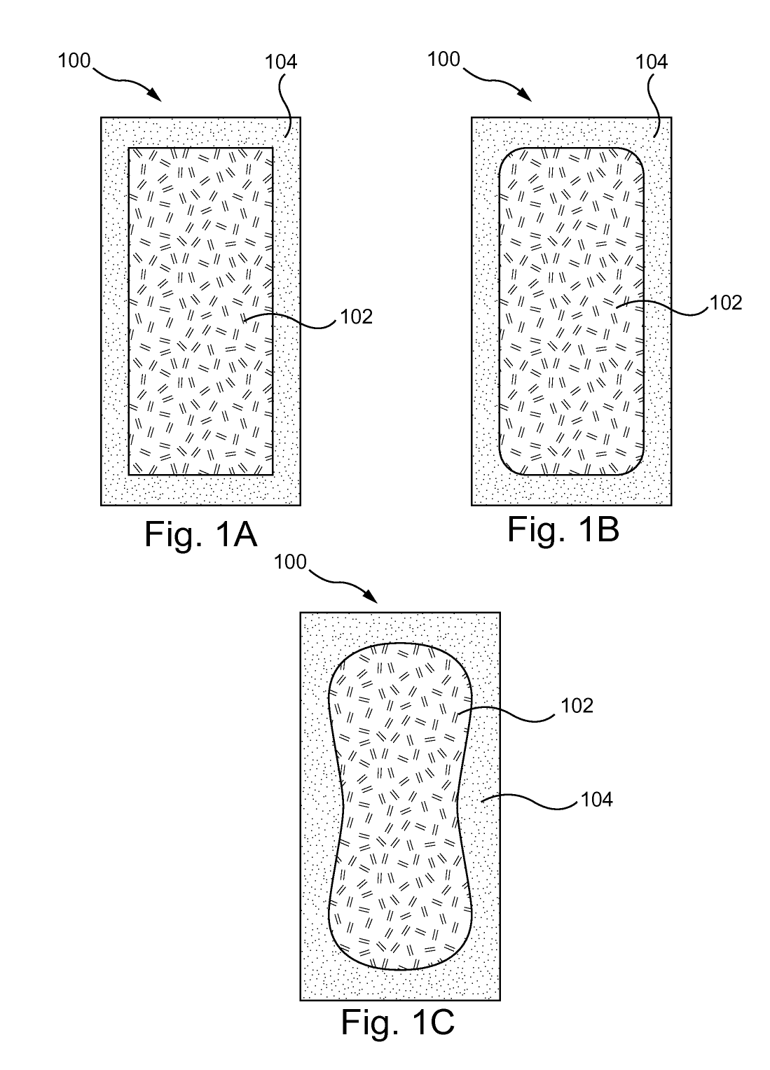

FIGS. 1A-1C are plan views of exemplary embodiments of absorbent articles 100, for example a diaper or sanitary pad. The absorbent article 100 can include the absorbent core 102 and the chassis 104. The absorbent core may have any desired shape. For example, as shown in FIGS. 1a-1c, the absorbent core 102 may be rectangular, rounded-rectangular, or may have an anatomically-conforming shape; however the absorbent core 102 should not be construed as being limited to solely the illustrated shapes.

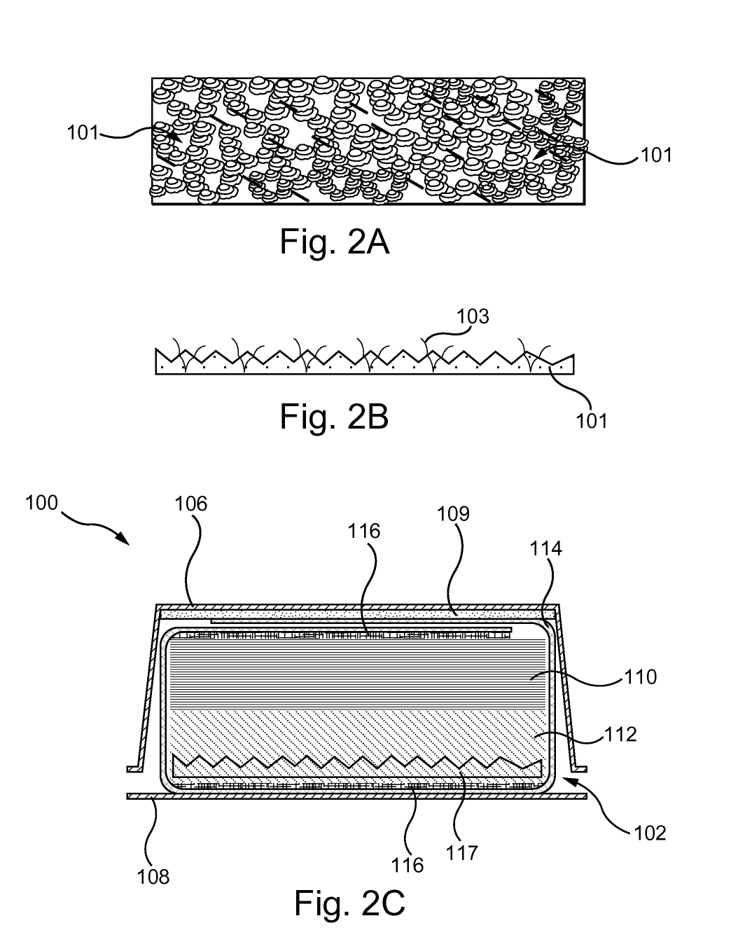

FIG. 2A is a top sectional view of an exemplary embodiment of a high loft layer showing the voids (open formation) 101 where the SAP may be stabilized and fixed.

FIG. 2B is a cross sectional view of an exemplary: embodiment of a high loft layer with fibers oriented substantially in Z direction 103, as would be understood by a person having ordinary skill in the art.

FIG. 2C is a cross sectional view of a typical absorbent article 100. The absorbent article 100 can include a permeable top sheet 106, a liquid impermeable back sheet 108 and the absorbent core 102 sandwiched therebetween. Additional layers, such as an acquisition layer 109, may be disposed between top sheet 106 and absorbent core 102 to facilitate faster liquid penetration into the core. The acquisition layer can be made of air-through bonded bi-component fibers or thermally bonded webs of polyester fibers. The absorbent core 102 can include a synthetic fiber matrix 110 positioned adjacent SAP 112, with both matrix 110 and SAP 112 enclosed by wrap 114, as well as the additional open formation bottom high loft layer with fibers oriented in a Z direction 117 to improve SAP stability. The SAP 112 and synthetic fiber matrix 110 and the open formation high loft layer with fibers oriented in a Z direction may be coupled to the wrap 114 by adhesive 116. The absorbent core 102 may be disposed between back sheet 108 and top sheet 106. Furthermore, absorbent core 102 can be positioned such that the synthetic fiber matrix is disposed between the permeable top sheet 106 and the SAP 112.

In some exemplary embodiments, the SAP 112, the top loft layer, synthetic fiber matrix 110 and the bottom loft layer can be contained within a wrap 114. Wrap 114 can be made of a material that can impede the passage of SAP 112 therethrough. Such materials can chide tissue, for example a single-ply white tissue having a high wet strength, and synthetic non-woven materials, for example polyolefin fibers such as polyethylene or polypropylene fibers.

Top sheet 106 may be any permeable polymeric non-woven sheet known in the art. A suitable top sheet 106 may be made from, for example, perforated plastic films, polyolefin fibers (e.g., polyethylene or polypropylene fibers), or combinations thereof.

Back sheet 108 may be any impermeable polymeric plastic and/or non-woven sheet known in the state of the art. For example, a suitable back sheet may be made from films of polyethylene, polypropylene, polyester, nylon, polyvinyl chloride or blends of these materials.

Absorbent core 102 may include SAP 112 in addition o a synthetic fiber matrix 110. The synthetic material used for matrix 110 can be, for example, polyolefins (e.g., polypropylene and polyethylene), rayon, polycarbonates, bicomponent fibers, cellulose acetate, and so forth. Such fibrous material is known in the art as TOW, and may be a crimped TOW. TOW is a continuous band composed of several thousand filaments, which can be held loosely together by a crimp, i.e, a wave configuration set into the band during its manufacture. The TOW band may be formed by combining the filaments from several spinnerets. The combined bundle of filaments can then be crimped to facilitate TOW band cohesion as well as suitable bulk and firmness when processed. Prior to making an absorbent composite that includes a TOW fiber, the TOW fiber can typically be unwound, opened, and then cut at various lengths and may be oriented in machine direction, so as to provide a fibrous mass of material. TOW having crimped filaments may be easier to open.

The TOW fiber can be any continuous or discontinuous thermoplastic filament TOW fiber that is capable of being opened and used in combination with SAP 112 in an absorbent core 102. The denier per fiber (dpf) of the TOW fiber can be in the range of about 1 dpf to about 9 dpf, for example about 5 dpf to about 8 dpf. For products having the same weight, filaments of lower dpf may provide increased surface area and increased moisture absorption. Total denier may vary within the range of about 26,000 denier to about 40,000 denier, depending upon the process used.

The SAP 112 may be polymers that can absorb and retain large amounts of a liquid relative to their own mass. A suitable SAP may be, for example, a sodium-neutralized cross-linked polyacrylate. Such a SAP may be formed by polymerization of a large quantity of units of acrylic acid blended with sodium hydroxide in the presence of an initiator. The acrylic acid and sodium hydroxide may be mixed together in the presence of water, an initiator, and a cross-linker. The resulting solution may undergo a polymerization process to form a three-dimensional polymer chain network, which may take the form of an aqueous gel. Subsequently, the aqueous gel may be chopped, crushed and dried to form SAP granules. The SAP granules may then be ground and sieved to obtain a desired particle size. At this point, the SAP granules may be further cross-linked so as to obtain desired absorbance under pressure characteristics.

The open formation bottom high loft layer may be made of air-through bonded fibers PET or PP or bi-component fibers PET/PE or PET/PP, with a basis weight varying between 40 gsm and 80 gsm and a width varying between 90 mm and 130 mm and a thickness varying between 1.5 mm and 4 mm.

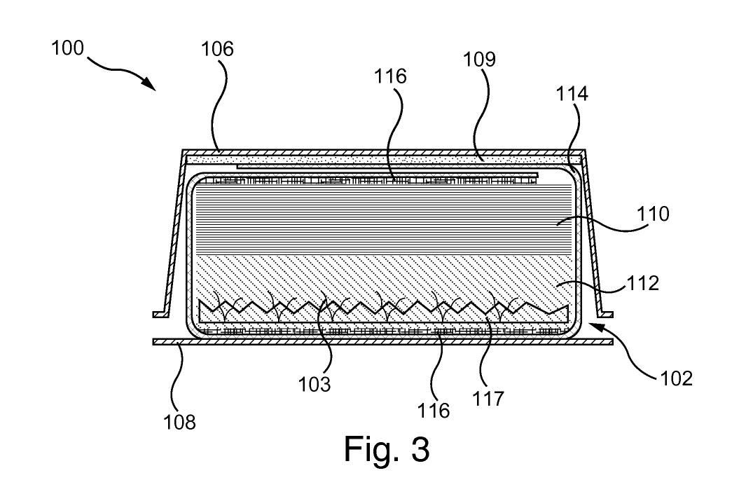

FIG. 3 illustrates exemplary positioning of the SAP 112 within the core 102 and its stabilization within the open formation bottom loft layer 117 with fibers 103 oriented in Z direction. The SAP 112 can be positioned such that, when core 102 is disposed within an absorbent article 100, the SAP 112 is proximate back sheet 108, while the synthetic fiber matrix is proximate top sheet 106. Both SAP 112 and matrix 110 can be coupled by an adhesive 116 to wrap 114.

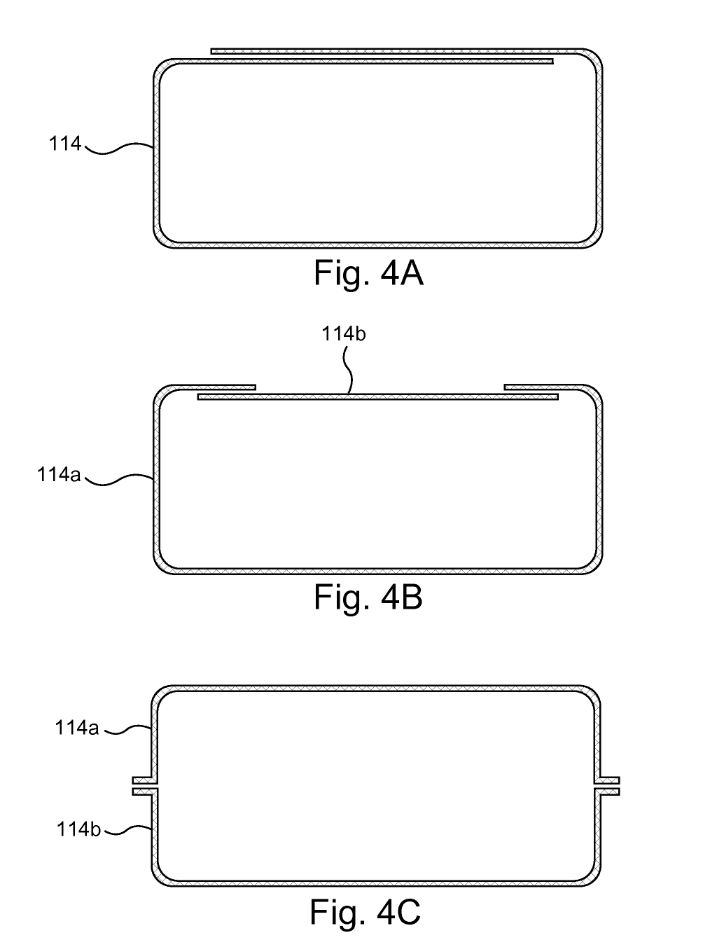

FIGS. 4A-4C illustrate exemplary configurations of wrap 114. As shown in FIG. 4A, wrap 114 may be provided as a single sheet of wrap, having a portion overlapping itself and coupled to itself. As shown in FIG. 4B, wrap 114 may be provided as two sheets of wrap, a first sheet 114a enclosing the bottom and sides of the absorbent core, and a second sheet 114b enclosing the top of the absorbent core and having a portion thereof overlapping and coupled to first sheet 114a. As shown in FIG. 4C, wrap 114 may be provided as two sheets of wrap, a first sheet 114a enclosing the top and sides of the absorbent core, and a second sheet 114b enclosing the bottom and sides of the absorbent core, with the two sheets 114a, 114b being coupled to each other at the sides of the absorbent core. The wrap may be formed in a variety of other configurations to achieve similar function, as would be understood by a person having ordinary skill in the art.

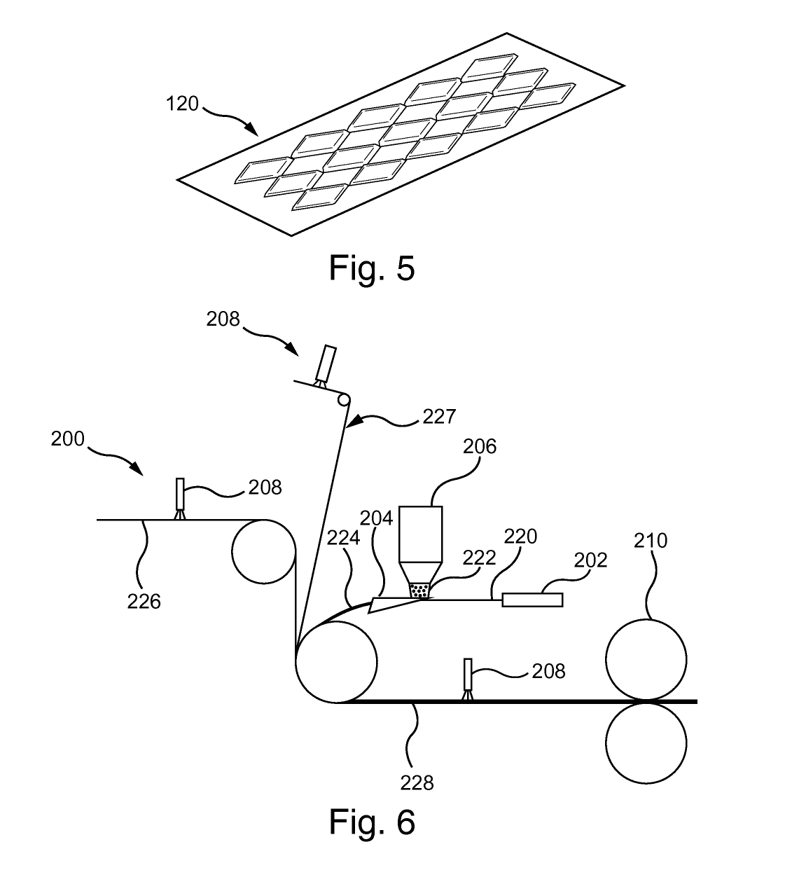

In yet other exemplary embodiments, the SAP 112 may be attached to the synthetic fiber matrix 110, the wrap 114, or the back sheet 108 and may be stabilized by the open formation bottom loft layer with fibers oriented in a Z direction. This can facilitate increasing the performance of the absorbent garment 100. Various chemical, mechanical, thermal or electrical means of attaching the SAP 112 to the synthetic matrix 110 may be employed. Any attachment capabilities may be suitable as long as it does not interfere with the ability of the SAP 112 to absorb liquid. Such capabilities include adhesives, heat sonication, embossing or sonic bonding patterns. It should also be understood that a combination of treatments may be used. FIG. 5 shows an exemplary embossed pattern 120 applied to a portion of an absorbent product. The embossed pattern 120 may subdivide the core 102 into pockets or cells containing SAP 112 and synthetic matrix 110. This can facilitate fixing the SAP 112 in place, thereby improving the performance of the absorbent article.

FIG. 6 illustrates an apparatus 200 for forming an exemplary absorbent article 100. The apparatus 200 can utilize any desired type of TOW fiber. The apparatus 200 can include a TOW feeder 202 that may be capable of feeding the opened TOW fiber 220 into a core forming station 204. A SAP feeder system 206 may provide SAP 222 to core forming station 204. The core forming station 204 may combine the TOW fiber 220 and SAP 222 to form an absorbent composite core 224. An adhesive may be applied on the open formation high loft layer with fibers oriented in a Z direction 227 by an applicator 208, covering the super absorbent polymer SAP and TOW at 224, the absorbent composite 224 may then be wrapped by a tissue or polyolefin layer 226, to which an adhesive can be applied by an applicator 208 forming a wrapped absorbent composite 228. Subsequently, the wrapped absorbent composite 228 can pass through an embossing station 210.

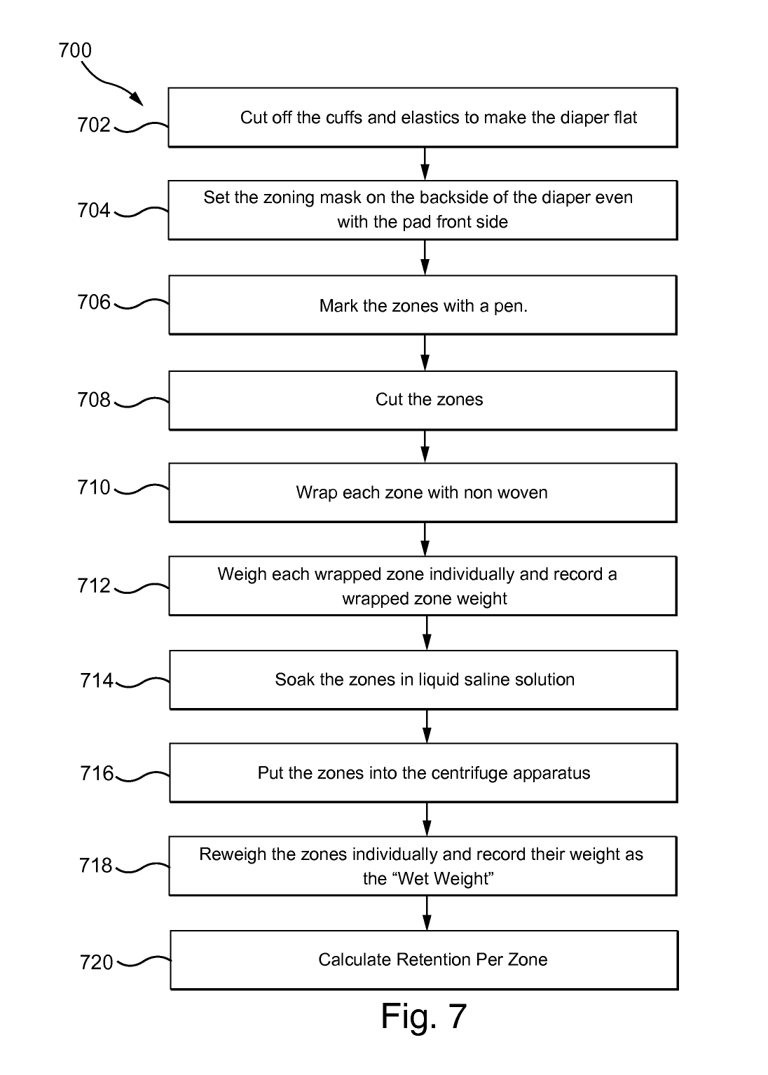

FIG. 7 illustrates an exemplary zoning testing 700 after shaking the absorbent article to measure the super absorbent polymer SAP distribution in the product through the retention per zone and to determine the basis weight of the product zones. A diaper may be used as an exemplary absorbent article in the present explanation.

The zoning testing 700 can include a digital scale with a precision of 0.01 g, container with hanging clips, stop wash timer, stapler and centrifuge apparatus with inner basket diameter of 28 cm, RPM 1400, scissors and zoning mask.

The test procedure may be as follows: In step 702, the cuffs and elastics may be cut off to make the diaper flat. In step 704, the zoning mask may be set on the backside of the diaper even with the pad front side. Next, in step 706, the zones may be marked with a pen. In an exemplary embodiment, there may be 7 zones. Zone 1 may be at the backside (tape level) and zone 7 may be at the front side (frontal tape level). In step 708, the zones may be cut using scissors. In step 710, each zone may be wrapped with non-woven. In an exemplary embodiment, the wrap may be secured with staples. Next, step 712, each wrapped zone may be weighed individually and the wrapped zone weight may be recorded. In step 714, the zones may be soaked in liquid saline solution for approximately 5 minutes. In step 716, the zones may be put into a centrifuge apparatus for approximately 1 minute. in step 718, the zones may be re weighed individually and their weight may he recorded as the “Wet Weight”. For the final step 720, the retention per zone may be calculated using the following formula:

Detention per zone=Wet Weight−Wrapped weight

The aqueous NaCl solution may be prepared as follows: approximately 9 g of sodium chloride crystals may be weighed and added along with approximately 1000 mL of de-ionized water to a clean dry flask, such as a 1000 mL flask. The concentration of the saline solution may be measured with a salt-meter to insure approximately 0.9% concentration.

The foregoing description and accompanying figures illustrate the principles, preferred embodiments and modes of operation of the invention. However, the invention should not be construed as being limited to the particular embodiments discussed above. Additional variations of the embodiments discussed above will be appreciated by those skilled in the art.

Therefore, the above-described embodiments should be regarded as illustrative rather than restrictive. Accordingly, it should be appreciated that variations to those embodiments can be made by those skilled in the art without departing from the scope of the invention as defined by the following claims.

What is claimed is:

1. An absorbent article, comprising:
a liquid permeable top sheet,
a pulpless absorbent core, the core including synthetic fibers over an open formation high loft layer having fibers in a Z direction and a super absorbent material positioned between the synthetic fibers and the open formation high loft layer, settled inside at least one void of the open formation high loft layer and between the fibers oriented in a Z direction of the open formation high loft layer; and
a liquid impermeable backsheet;
wherein the pulpless absorbent core is positioned between the top sheet and the backsheet.

2. The absorbent article of claim 1, wherein the super absorbent material is settled inside the at least one void and between the fibers oriented in a Z direction of the open formation high loft layer and TOW fibers.

3. The absorbent article of claim 1, wherein the super absorbent material is a sodium neutralized, cross-linked polyacrylate.

4. The absorbent article of claim 1, wherein the absorbent core further comprises a wrap.

5. The absorbent article of claim 4, wherein the wrap is formed from one of a polyolefin, a tissue, and a synthetic non-woven material.

6. The absorbent article of claim 1, wherein the synthetic fibers are TOW fibers selected from the group consisting of cellulose acetate fibers, polypropylene fibers, rayon fibers, polyacrylonitrile fibers, polypropylene and polyethylene bicomponent fibers, cotton fibers and cotton linter fibers.

7. The absorbent article of claim 1, wherein the synthetic fibers are white.

8. The absorbent article of claim 1, wherein the synthetic fibers are colored.

9. The absorbent article of claim 1, wherein the synthetic fibers are perfumed.

10. The absorbent article of claim 1, wherein the super absorbent material is settled inside the at least one void and between the fibers oriented in a Z direction attic open formation high loft layer and the synthetic fibers, the super absorbent material is coupled to at least one of the synthetic fibers and the wrap, and the super absorbent material is coupled by at least one of a chemical coupling and a mechanical coupling.

11. The absorbent article of claim 10, wherein the chemical coupling is an adhesive.

12. The absorbent article of claim 10, wherein the mechanical coupling is a heated embossing.

13. The absorbent article of claim 1, wherein the absorbent core further comprises a top acquisition layer and a bottom high loft layer with a weight from 40 gsm to 80 gsm.

14. The absorbent article of claim 1, wherein the super absorbent material is 50% to 95% by weight of the absorbent core.

15. The absorbent article of claim 1, wherein a portion of the absorbent article is embossed. such that the absorbent core is subdivided into pockets or cells.

16. A method of zoning testing an absorbent article comprising:
removing excess material of an absorbent article to make it flat;
setting a zoning mask on the backside of the article, even with a front side of a pad;
marking zones on the article with a pen;
cutting the zones using scissors;
wrapping each zone with a nonwoven using staples;
weighing each wrapped zone individually and recording a wrapped weight;
soaking the zones in liquid saline solution for approximately minutes;
putting the zones into a centrifuge apparatus for 1 minute;
reweighing the zones individually and recording their weight as a “Wet Weight”; and
calculating retention per zone by subtracting the Wrapped Weight from the Wet Weight.
